(19) United States
(12) Patent Application Publication
(10) Pub. No.: US 2023/0310005 A1
(43) Pub. Date: Oct. 5, 2023

(54) SYSTEMS AND METHODS FOR EMBOLIC IMPLANT DEPLOYMENT

(71) Applicant: DePuy Synthes Products, Inc., Raynham, MA (US)

(72) Inventors: Zhixian CHEN, Raynham, MA (US); Daniel SOLAUN, Miami, FL (US); David BLUMENSTYK, Miami, FL (US)

(73) Assignee: DePuy Synthes Products, Inc., Raynham, MA (US)

(21) Appl. No.: 17/707,130

(22) Filed: Mar. 29, 2022

(51) Int. Cl. A61B 17/12

(52) U.S. Cl. CPC A61B 17/1214; A61B 2017/12054; A61B 17/12113

(57) ABSTRACT

Disclosed herein are various exemplary systems, devices, and methods for delivering an implantable medical device to a target location of a body vessel. The system can include a generally hollow deliver tube that includes a lumen therethrough, a proximal end, and a distal end. An implant can be disposed on the distal end, and can have a proximal end having a plurality of engagement grooves. A pull member can be disposed within the lumen with a coiled distal end engaged to the engagement grooves. The pull member can be configured to disengage from the engagement grooves in response to being rotated axially or being translated proximally. In another aspect, the implant can have a notch member on a proximal end, and the pull member can include a distal end that is shaped to engage the notch member and disengage in response to the pull member being rotated axially.

### FIELD OF INVENTION

The present invention relate to aneurysm treatment devices and more particularly, to improved delivery systems for embolic implants.

### BACKGROUND

Numerous intravascular implant devices are known in the field. Many are deployed mechanically, via systems that combine one or more catheters and wires for delivery. Examples of implants that can be delivered mechanically include embolic elements, stents, grafts, drug delivery implants, flow diverters, filters, stimulation leads, sensing leads, or other implantable structures delivered through a microcatheter. Some obstetric and gastrointestinal implants may also be implanted via similar systems that combine one or more catheters and wires. Devices that may be released or deployed by mechanical means vary greatly in design but can employ a similar delivery catheter and wire system. Many such catheter-based delivery systems include a wire for retention of the implant in the catheter until the time for release of the device. These systems are then actuated by retracting or pulling the wire relative to the catheter. Such a wire is referred to herein as a “pull wire”.

One issue with current catheter-based delivery systems is premature detachment of the implantable device. Premature detachment occurs when the implant is detached from the delivery system before reaching the treatment site. This may occur due to the tortuosity experienced by the delivery system as it passes through the vasculature of the patient, which can cause an increase in friction between the “pull wire” and the delivery system causing the pull wire to move proximally while the delivery system is moving distally.

Accordingly, there is a need for an improved implant delivery system that prevents premature detachment of the implant as it is delivered through tortuous vasculature. This disclosure is directed to this and other considerations.

### SUMMARY

Disclosed herein are various exemplary systems, devices, and methods for delivering an implantable medical device to a target location of a body vessel. The system can include a generally hollow deliver tube that includes a lumen therethrough, a proximal end, and a distal end. An implant can be disposed on the distal end, and can have a proximal end having a plurality of engagement grooves. A pull member can be disposed within the lumen with a coiled distal end engaged to the engagement grooves. The pull member can be configured to disengage from the engagement grooves in response to being rotated axially or being translated proximally. In another aspect, the implant can have a notch member on a proximal end, and the pull member can include a distal end that is shaped to engage the notch member and disengage in response to the pull member being rotated axially.

In one aspect, a detachment system for delivering an implantable medical device to a target location of a body vessel is disclosed. The detachment system can include a generally hollow delivery tube, which can include a lumen therethrough, a proximal end, and a distal end. The detachment system can include an implant disposed on the distal end of the delivery tube. The implant can have a proximal end which includes a plurality of engagement grooves. The detachment system can include a pull member that is disposed within the lumen. The pull member can have a coiled distal end that is engaged to the engagement grooves. The pull member can be configured to disengage from the engagement grooves and cause the implant to be released in response to the pull member being rotated axially or being translated proximally.

In some embodiments, the detachment system can include a compressed portion of the delivery tube that is between the proximal end and the distal end. The compressed portion can be axially movable from a compressed state to an elongated state. The pull member can be under tension and the compressed portion can be configured to automatically move from the compressed state to the elongated state in response to the pull member disengaging from the engagement grooves.

In some embodiments, the compressed portion of the delivery tube can include a spiral-cut portion of the delivery tube.

In some embodiments, the proximal end of the implant can include a cylindrical shape. In some embodiments, the proximal end of the implant can include a rectangular prism configuration. In some embodiments, the implant can include an embolic coil. In some embodiments, the pull member coiled distal end is wrapped around the implant. In some embodiments, the pull member can be a wire. In some embodiments, the coiled distal end of the pull member is preformed to the engagement grooves of the implant.

In another aspect, a detachment system for delivering an implantable medical device to a target location of a body vessel is disclosed. The detachment system can include a generally hollow delivery tube that includes a lumen, a proximal end, a distal end, and a compressed portion of the delivery tube. The compressed portion of the delivery tube can be located between the proximal end and the distal end, and the compressed portion can be axially movable from a compressed state to an elongated state. The detachment system can include an implant disposed on the distal end of the delivery tube. The implant can have a proximal end which includes a notch member. The detachment system can include a pull member disposed within the lumen. The pull member can include a distal end that is shaped to engage the notch member while the compressed portion of the delivery tube is in the compressed state. The pull member can be configured to be rotated axially to be disengaged from the notch member and cause the compressed portion to move from the compressed state to the elongated state.

In some embodiments, the pull member is at a first position of axial rotation and the distal end of the pull member and the notch member are engaged to each other with an interference engagement. The pull member can be configured to be axially rotated to a second position with respect to the notch member so that the distal end of the pull member is disengaged from the notch member.

In some embodiments, a rotation angle between the first position and the second position can measure approximately 90 degrees. In some embodiments, the compressed portion of the delivery tube can include a spiral-cut portion of the delivery tube.

In some embodiments, the implant can include an embolic coil. In some embodiments, the distal end of the pull member can include a protrusion shaped to engage the notch member and rotate out of the notch member in response to the pull member being rotated axially.

In some embodiments, the tension of the pull member maintains the compressed portion in the compressed state and the disengagement of the pull member from the notch member allows the compressed portion to move from the compressed state to the elongated state and apply a force distally to the implant.

In some embodiments, the tension applied to the pull member due to compression of the compressed portion facilitates the movement of the protrusion from the notch member.

In some embodiments, the proximal end of the implant is at least partially disposed within the lumen of the delivery tube.

In another aspect, a method of detaching an implantable medical device. The medical device can include a hollow delivery tube having a proximal end and a distal end. The method can include providing a compressed portion of the delivery tube between the proximal end and the distal end. The method can include engaging the implantable medical device to a coiled distal end of a pull member, the pull member disposed in a lumen of the delivery tube while the compressed portion is in a compressed state. The method can include translating a pull member proximally, thereby unraveling the coiled distal end of the pull member from the implantable medical device while allowing the compressed portion to extend from the compressed state to an elongated state to release implantable medical device.

In some embodiments, a force required to translate the pull member proximally to disengage the coiled distal end of the pull member from the implantable medical device is between approximately 1 Newton and approximately 4 Newtons.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIGS. 1A-1C are illustrations of a delivery system and implant, according to aspects of the present invention.

FIGS. 2A-2C are illustrations of another delivery system and implant, according to aspects of the present invention.

FIGS. 3A-3D are illustrations of another delivery system and implant, according to aspects of the present invention.

FIG. 4 is a flowchart of an example method of using the delivery system, according to aspects of the present invention.

### DETAILED DESCRIPTION

The following description of certain examples of the invention should not be used to limit the scope of the present invention. The drawings, which are not necessarily to scale, depict selected embodiments and are not intended to limit the scope of the invention. The detailed description illustrates by way of example, not by way of limitation, the principles of the invention. Other examples, features, aspects, embodiments, and advantages of the invention will become apparent to those skilled in the pertinent art from the following description, which is by way of illustration, one of the best modes contemplated for carrying out the invention. As will be realized, the invention is capable of other different or equivalent aspects, all without departing from the invention. Accordingly, the drawings and descriptions should be regarded as illustrative in nature and not restrictive.

Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the pertinent art in view of the teachings herein. Such modifications and variations are intended to be included within the scope of the claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

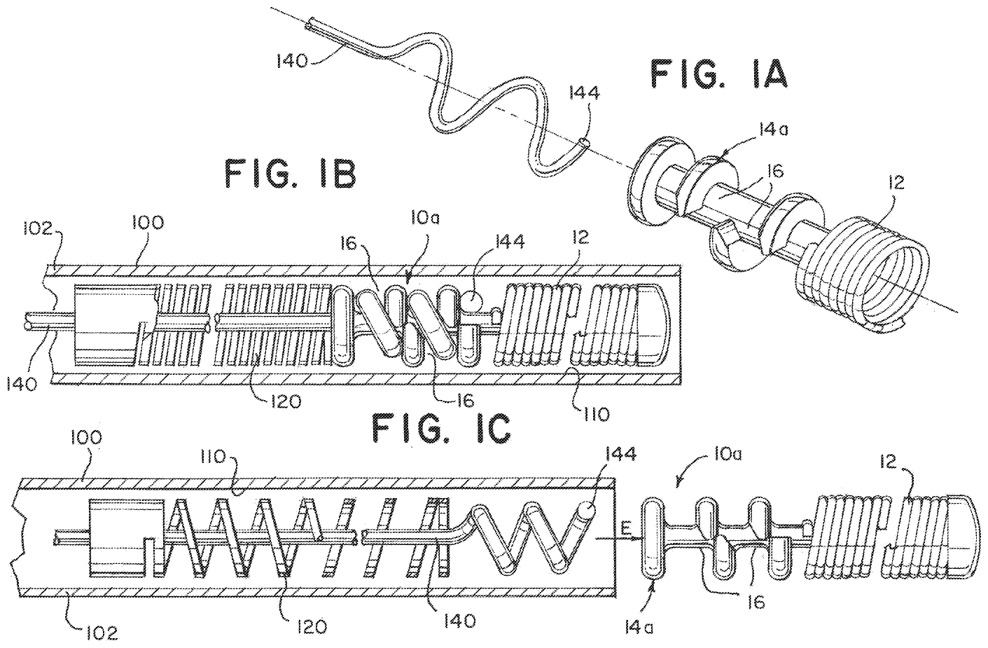

Turning to the figures, as illustrated in FIGS. 1A-1C, an example delivery system 10a is shown. The delivery system can include a generally hollow delivery tube 100 that has a proximal end 102 and a distal end 104. Within the delivery tube 100 is a lumen 110 therethrough. Disposed within lumen 110 can be a pull member 140. The pull member 140 can have a distal end 144, which can have a generally coiled (e.g., corkscrew) shape. Located proximal to the distal end 144 of the pull member 140 can be an implant 12. Implant 12 can include a proximal end 14a. The proximal end 14a of the implant 12 can have grooves 16 shaped to mate with distal end 144 of the pull member 140. In some embodiments, coiled distal end 144 can be configured in a corkscrew shape that can be preformed to interface with grooves 16 of implant 12. As shown in FIG. 1A, implant 12 can have a generally flat, rectangular shape, with the addition of grooves 16 that allow implant 12 to mate with distal end 144 of pull member, although other shapes of implant 12 are not precluded, as would be understood by a person having skill in the pertinent art. According to some embodiments, implant 12 can be an embolic coil. Grooves 16 of implant 12 can be understood as areas in which material from proximal end 14a of implant 12 has been selectively removed to form recesses within the material of proximal end 14a of implant 12. Grooves 16 can be contrasted with apertures, as apertures are characterized by an area in which no material is present or an area in which all material has been completely removed as opposed to a groove, which can be characterized by at least some material is left after a groove 16 is formed.

Delivery system 10a can include a compressible portion 120. The compressible portion 120 can be axially adjustable between an elongated condition and a compressed condition. The compressible portion 120 can be formed from a spiral-cut portion of the delivery tube 100, formed by a laser cutting operation. Additionally, or alternatively, the compressible portion can be formed of a wound wire, spiral ribbon, or other arrangement allowing axial adjustment according to the present invention. Preferably, compressible portion 120 is in the elongated condition at rest and automatically or resiliently returns to the elongated condition from a compressed condition, unless otherwise constrained.

As shown in FIGS. 1A-1C, pull member 140 can be rotated axially in order to attach and detach the distal end 144 of pull member 140 to implant 12. For example, the pull member 140 can be rotated axially in a first direction (e.g., clockwise) which allows the coiled (e.g., corkscrew shaped) distal end 144 of pull member 140 to engage to grooves 16 of implant 12. FIG. 1B shows the coiled distal end 144 of pull member 140 engaged to the engagement grooves 16 of implant 12.

FIG. 1B shows delivery system 10a in which distal end 144 of pull member 140 is attached to grooves 16 of implant 12. Distal end 144 of pull member 140 can be detached from implant 12 in a similar manner as it is attached to the implant 12. For example, pull member 140 can be rotated in a second direction (e.g., counterclockwise) which allows the coiled distal end 144 of pull member 140 to disengage from grooves 16 of implant 12, thereby facilitating release of implant 12 from the delivery system 10a. Alternatively, an operator could pull pull member 140 proximally, in which case the coiled distal end 144 can be caused to unravel from engagement grooves 16 of implant 12 until implant 12 is released from coiled distal end 144. The force necessary to rotate the pull member 140 axially to disengage the implant from the pull member 140 can be between approximately 1 Newton and approximately 2 Newtons. The force necessary to cause the distal end 144 of pull member 140 to unravel from grooves 16 of implant 12 due to proximal translation of pull member 140 can be between approximately 1 Newton and approximately 4 Newtons. FIG. 1C shows the delivery system 10a at the moment the implant 12 is released from the distal end 144 of pull member 140. As discussed above, implant 12 may be deployed based on axial rotation of pull member 140 and/or proximal translation of pull member 140. As seen in FIG. 1C, the compressible portion 120 can be configured to expand from a compressed configuration to an elongated configuration as the implant 12 is detached, thereby providing an elastic force against implant 12 to facilitate deployment to a treatment site.

According to some embodiments, the delivery system 10a can be configured to delivery implant 12 through a patient's vasculature to a treatment site, such as into an aneurysm.

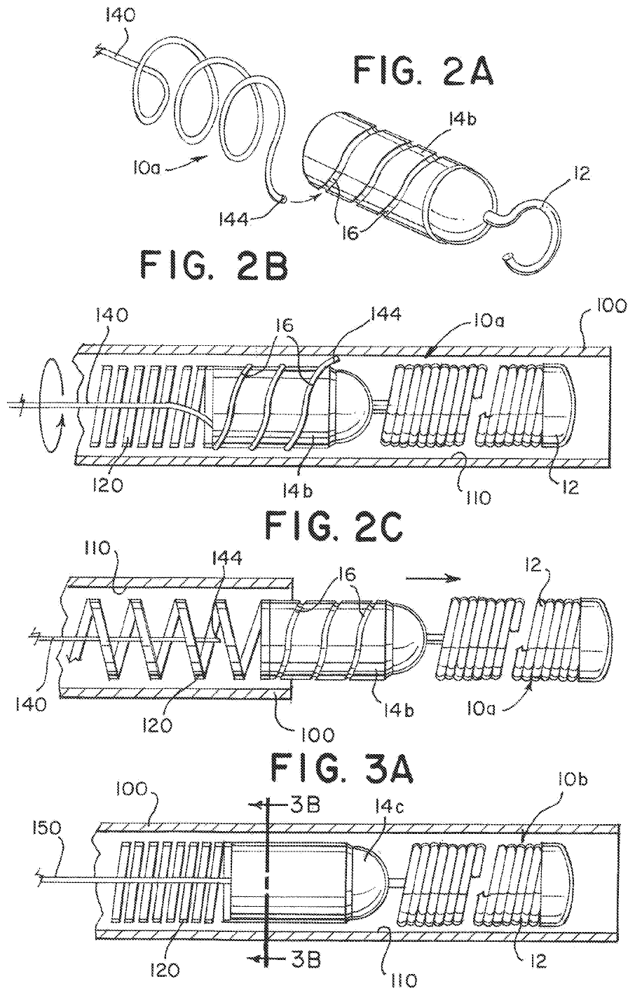

Turning to FIGS. 2A-2C, an alternative embodiment of delivery system 10a is shown. In the alternative embodiment, the delivery system 10a can include a pull wire 140 with a coiled (e.g., corkscrew shaped) distal end 144, a generally hollow delivery tube 100 with a lumen 110 therethrough, and an implant 12 having a proximal end 14b. Unlike proximal end 14a shown in FIGS. 1A-1C, proximal end 14b may have a generally cylindrical shape. Proximal end 14b may additionally include a plurality of engagement grooves 16 cut into proximal end 14b. Similarly to proximal end 14a, the grooves 16 of proximal end 14b can be contrasted with apertures, as grooves 16 are characterized with at least a portion of material remaining within the recesses that form grooves 16. The pull member 140 can similarly be rotated axially in order to engage and/or disengage the coiled distal end 144 of pull member 140 to/from implant 12. As shown in FIGS. 1A-1C and 2A-2C, the coiled distal end 144 of pull member 140 can be preformed or pre-shaped to engage with the engagement grooves 16 of implant 12.

FIGS. 3A-3D collectively depict an alternative delivery system 10b. Delivery system 10b include a generally hollow delivery tube 100 having a lumen 110 therethrough. Disposed within lumen 110 is a pull member 150, which may include a distal end 154. Located distal to the pull member distal end 154 can be an implant 12. Implant 12 can have a proximal end 14c. Unlike proximal ends 14a, 14b, proximal end 14c may not include engagement grooves 12 in the surface of the implant 12. Instead, proximal end 14c may include a notch 18 which is configured to engage with distal end 154 of pull member 150. That is, distal end 154 can have a shape configured to fit tightly into notch 18 in order to secure implant 12 to delivery system 10b.

FIG. 3A shows delivery system 10b with the pull member 150 engaged to implant 12 via notch 18 interfacing with distal end 154 of pull member 150. In order to attach distal end 154 to implant 12, pull member 150 may be rotated axially until the distal end 154 of pull member 150 is aligned with the notch 18 of implant 12. Once aligned, distal end 154 of pull member 150 may tightly fit within notch 18 of pull member 12 as shown in FIGS. 3B and 3C, at which point the pull member 150 may be rotated such that distal end 154 is placed out of alignment with notch 18, thereby securing the pull member 150 to the implant 12 via notch 18. That is, after distal end 154 is positioned inside notch 18, pull member 150 can be rotated axially which positions distal end 154 such that distal end 154 interferes with notch 18 and secures implant 12 to delivery system 10b. Delivery system 10b with implant 12 attached to pull member 150 can be seen in FIG. 3A, with distal end 154 rotated out of alignment with notch 18 to secure implant 12. According to some embodiments, compressible portion 120 can be placed in compression prior to attaching implant 12 to pull member 150. FIG. 3D illustrates delivery system 10b at the moment the pull member 150 is rotated axially to thereby release the implant 12 from the delivery system 10b. As shown, compressible portion 120 of delivery tube can move along a longitudinal axis from a compressed configuration to an elongated configuration, thereby providing an elastic force E against the implant.

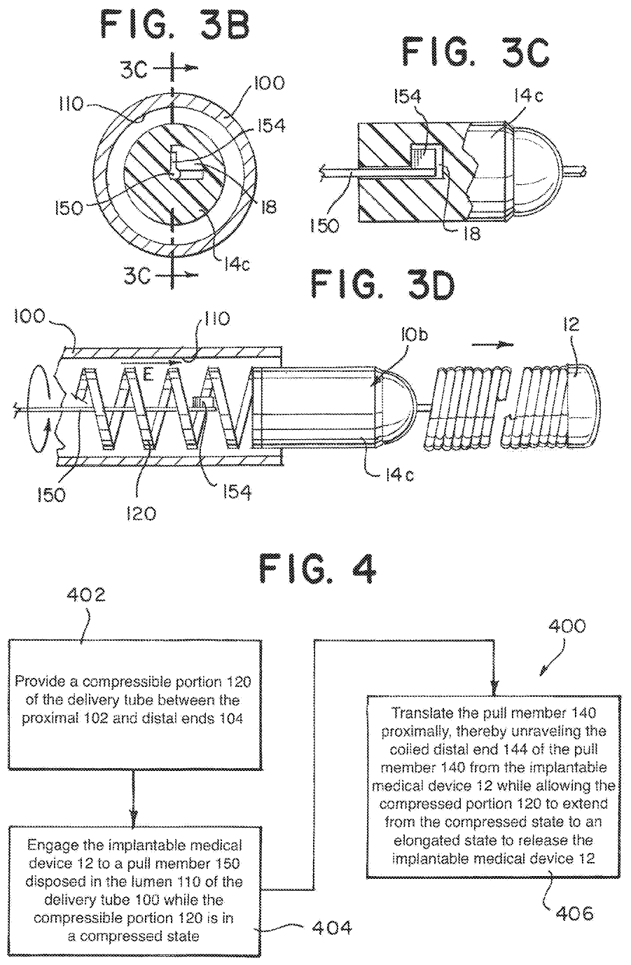

FIG. 4 is a flowchart showing an exemplary method of operating a delivery system 10a, 10b, according to aspects of the present invention. In step 402, the method includes providing a compressible portion 120 of delivery tube 100 between proximal end 102 and distal end 104. In step 404, the method includes engaging an implantable medical device 12 to a pull member 140 disposed in the lumen 110 of delivery tube 100 while the compressible portion 120 is in a compressed configuration. As described with respect to FIGS. 1A-1C and 2A-2C, pull member 140 can be engaged to implant 12 by rotating the pull member 140 axially, which allows coiled distal end 144 to wrap around grooves 16 of implant 12 until implant 12 is secured to pull member 140.

In step 406, the method includes rotating the pull member 150 axially to thereby disengage the pull member 150 from the implantable medical device. As pull member 150 is disengaged from implant 12, compressible portion 120 is allowed to extend from a compressed configuration to an elongated configuration, which imparts an elastic force against implant 12, which facilitates a successful deployment of implant 12 to a treatment site.

1. A detachment system for delivering an implantable medical device to a target location of a body vessel, comprising:
a generally hollow delivery tube comprising:
a lumen therethrough,
a proximal end, and
a distal end,
an implant disposed on the distal end of the delivery tube, the implant having a proximal end comprising a plurality of engagement grooves;
a pull member disposed within the lumen, the pull member comprising a coiled distal end engaged to the engagement grooves;
wherein the pull member is configured to disengage from the engagement grooves and cause the implant to be released in response to being rotated axially or being translated proximally.

2. The detachment system of claim 1, further comprising a compressed portion of the delivery tube between the proximal end and distal end which is axially movable from a compressed state to an elongated state, wherein the pull member is under tension, and wherein the compressed portion is configured to automatically move from the compressed state to the elongated state in response to the pull member disengaging from the engagement grooves.

3. The detachment system of claim 2, wherein the compressed portion of the delivery tube comprises a spiral-cut portion of the delivery tube.

4. The detachment system of claim 1, wherein the proximal end of the implant comprises a cylindrical shape.

5. The detachment system of claim 1, wherein the proximal end of the implant comprises a rectangular prism configuration.

6. The detachment system of claim 1, wherein the implant comprises an embolic coil.

7. The detachment system of claim 1, wherein the pull member coiled distal end is wrapped around the implant.

8. The detachment system of claim 1, wherein the pull member comprises a wire.

9. The detachment system of claim 1, wherein the coiled distal end of the pull member is preformed to the engagement grooves of the implant.

10. A detachment system for delivering an implantable medical device to a target location of a body vessel, comprising:
a generally hollow delivery tube comprising:
a lumen therethrough,
a proximal end,
a distal end, and
a compressed portion of the delivery tube between the proximal end and distal end which is axially movable from a compressed state to an elongated state;
an implant disposed on the distal end of the delivery tube, the implant having a proximal end) comprising a notch member;
a pull member disposed within the lumen, the pull member comprising a distal end shaped to engage the notch member while the compressed portion is in the compressed state;
wherein the pull member is configured to be rotated axially to thereby disengage from the notch member and cause the compressed portion to move from the compressed state to the elongated state.

11. The detachment system of claim 10,
wherein the pull member is at a first position of axial rotation and the distal end of the pull member and the notch member are engaged to each other with an interference engagement, and
wherein the pull member is configured to be axially rotated to second position with respect to the notch member so that the distal end of the pull member is disengaged from the notch member.

12. The detachment system of claim 11, wherein a rotation angle between the first position and the second position measures approximately 90 degrees.

13. The detachment system of claim 10, wherein the compressed portion of the delivery tube comprises a spiral-cut portion of the delivery tube.

14. The detachment system of claim 10, wherein the implant comprises an embolic coil.

15. The detachment system of claim 10, wherein the distal end of the pull member comprises a protrusion shaped to engage the notch member and rotate out of the notch member in response to the pull member being rotated axially.

16. The detachment system of claim 10,
wherein tension in the pull member maintains the compressed portion in the compressed state, and
wherein disengagement of the pull member from the notch member allows the compressed portion to move from the compressed state to the elongated state and apply a force distally to the implant.

17. The detachment system of claim 15, wherein tension applied to the pull member due to compression of the compressed portion facilitates movement of the protrusion from the notch member.

18. The detachment system of claim 10, wherein the proximal end of the implant is at least partially disposed within the lumen of the delivery tube.

19. A method of detaching an implantable medical device, comprising a hollow delivery tube having a proximal end and a distal end comprising the steps of:
providing a compressed portion of the delivery tube between the proximal and distal ends;
engaging the implantable medical device to a coiled distal end of a pull member disposed in a lumen of the delivery tube while the compressed portion is in a compressed state; and
translating the pull member proximally, thereby unraveling the coiled distal end of the pull member from the implantable medical device while allowing the compressed portion to extend from the compressed state to an elongated state to release the implantable medical device.

20. The method of claim 19, wherein a force required to translate the pull member proximally to disengage the coiled distal end of the pull member from the implantable medical device is between approximately 1 Newton and approximately 4 Newtons.
